(19) United States
(12) Patent Application Publication
(10) Pub. No.: US 2015/0225756 A1
(43) Pub. Date: Aug. 13, 2015

(54) ALCOHOL SULFITE BIOREFINERY PROCESS

(71) Applicant: API Intellectual Property Holdings, LLC, Atlanta, GA (US)

(72) Inventors: Theodora RETSINA, Atlanta, GA (US); Vesa PYLKKANEN, Atlanta, GA (US)

(21) Appl. No.: 14/693,495

(22) Filed: Apr. 22, 2015

Related U.S. Application Data
(63) Continuation of application No. 13/500,916, filed on Apr. 9, 2012.
(60) Provisional application No. 61/250,082, filed on Oct. 9, 2009.

(51) Int. Cl. C12P 19/14; C12P 19/02

(52) U.S. Cl. CPC C12P 19/14; C12P 19/02

(57) ABSTRACT

A biorefinery process to fractionate lignocellulosic materials into cellulose, hemicelluloses and lignin using a pretreatment with mixture of alcohol, sulfur dioxide and water. Further treatment with enzymes, micro-organisms, and optionally bisulfite ion, are used to convert intermediate products to alcohol and lignin derivatives.

### FIELD OF THE INVENTION

This invention describes an integrated biorefinery process, where lignocellulosic material is converted to bioalcohol, cellulose, and lignin derivatives. In particular, alcohol sulfite pretreatment is applied to separate cellulose fibers, dissolve lignin and hemicelluloses. Enzymes are used to complete sugar hydrolysis. Pentose and hexose sugar utilizing micro-organisms are employed in the fermentation process.

### BACKGROUND OF THE INVENTION

Two current biorefinery technologies are prevalent, thermal and biochemical methods. Gasification and pyrolysis are thermal methods to obtain building blocks for the biofuels and chemicals. The biochemical methods rely on chemicals and micro-organisms to break down lignocellulosic material into fermentable sugars.

The biochemical methods typically include pretreating lignocellulosic material into accessible fragments, post hydrolysis, and fermentation of sugars. Lignin is preferably removed and combusted for the process energy. The hemicelluloses consist of sugars that cannot be easily fermented using commercial micro-organisms. Therefore a clean fractionation of the lignocellulosic components in one or more steps is desirable.

Sulfite pulping was early commercial fractionation technology to produce cellulose, ethanol and lignosulfonate. The low solubility of sulfur dioxide in water and slow diffusion of water to wood chips necessitate the use of counter ions and several hours of cooking time. Sulfite spent liquors that contain the counter ion, lignin and hemicelluloses throughout the recovery of ethanol result in relatively low yields. After a removal of ethanol, the remaining cooking chemicals and lignin are either burned or sold as lignosulfonates bound with calcium, magnesium, sodium and ammonia counter ion.

Fractionation using solvent or solvents have been proposed to produce cellulose, lignin and hemicelluloses free of cooking chemicals. The solvents proposed, absent of sulfur based catalyst, are not effective in dissolving softwood lignin. Ethanol solvent, in particular, requires high temperature and pressure to effectively dissolve even hardwood lignin.

The original solvent process is described in U.S. Pat. No. 1,856,567 by Kleinert et al. Although three demonstration size facilities: ethanol-water (ALCELL™); alkaline sulfite with anthraquinone and methanol (ASAM™); and ethanol-water-sodium hydroxide (Organocell™) were operated briefly in the 1990's, there are no full scale solvent pulp mills today. Only ALCELL™ produced significant byproduct, namely native reactive lignin, from the spent pulping liquor.

Groombridge et al. in U.S. Pat. No. 2,060,068 shows that an aqueous solvent with sulfur dioxide is a potent delignifying system to produce cellulose from lignocellulosic material. Their process was limited to 9% concentration of sulfur dioxide in the liquid phase.

Finally, in U.S. Pat. No. 5,730,837 to Black et al. describes liquid phase fractionation of lignocellulosic material into lignin, cellulose and dissolved sugars using ketone, alcohol, water and mineral acid. This is more readily known as the NREL clean fractionation technology. The separation of lignin and sugars in two immiscible layers are noted. The lignin-ketone layer requires its own recovery cycle for lignin purification.

The present inventors have developed an integrated biorefinery process, where heated aqueous alcohol and sulfur dioxide are used to rapidly dissolve lignin and hemicelluloses from wood. Alcohol strength of 30% or more and sulfur dioxide of 9% or more is used. The process further cleans cellulose, recovers sulfur dioxide and alcohol from the spent liquor, and separates lignin. The cellulosic sugars are enzymatically hydrolyzed and fermented using commercial micro-organisms. The hemicellulosic sugars are autohydrolyzed in the lignosulfonic acid, which was formed during the cooking, and the sugars are fermented with a capable micro-organism.

Therefore, in the prior art of fractionating lignocellulosic material:
a) The sulfite processes, where low sulfur dioxide charge results in slow reaction rate and the requirement of the counter ions.
b) Ethanol pulping, where high temperature are used to speed reaction rate, but does not dissolve softwood lignin.
c) Multi-solvent pulping, where each solvent requires its own recovery cycle.
   The current inventors developed a process that is both rapid and offers simple, efficient recovery of the cooking chemicals. This is achieved through cooking lignocellulosic material with sulfur dioxide and alcohol in a continuous process.

### BRIEF SUMMARY OF THE INVENTION

The present invention describes a process of fractionating lignocellulosic material into lignin, cellulose and hemicelluloses in cooking with water, alcohol and sulfur dioxide. The cooked material is washed counter currently to remove cooking chemicals, lignin and dissolved hemicelluloses, while the remaining cellulose is further enzymatically processed to alcohol. The spent liquor is freed of cooking chemicals, lignin is separated and hemicelluloses are fermented to alcohol, and soluble lignosulfonate recovered after alcohol distillation.

### BRIEF DESCRIPTION OF THE DRAWINGS

A more complete understanding of the present invention may be obtained by reference to the following detailed description, when read in conjunction with the accompanying drawing wherein:

FIG. 1. Illustrates a flow sheet example of the biorefinery process, noting that the process steps may be in other sequences.

### DETAILED DESCRIPTION OF THE INVENTION

A biorefinery process to convert lignocellulosic material into alcohol and lignin derivatives through vapor phase cooking of lignocellulosic material with alcohol, water, and sulfur dioxide comprising the steps of:
1) Charging lignocellulosic material such as wood chips in to a pressurized cooking vessel, and optionally, using a dewatering device.
2) Charging the cooking vessel with water, sulfur dioxide and alcohol.
3) Heating the contents of the vessel with direct or indirect steam.
4) Pumping or blowing the digested lignocellulosic material through a dilution valve to convert it to cellulose.
5) Washing the cellulose in several countercurrent steps. In one manifestation, an alcohol stripper is integrated to treat one or more of the washing filtrates to remove alcohol and reuse distillation bottoms for washing. This allows a high apparent dilution factor in that stage with low overall water usage.
6) Hydrolyzing the washed cellulose using enzymes to monomeric sugars.
7) Fermenting the cellulosic hydrolyzate to dilute cellulosic alcohol.
8) Distilling the dilute cellulosic alcohol. In one manifestation the distillation steps are occurring at low temperature and the enzymes remain active. In this case the bottoms of the distillation column are returned to be used as dilution in the enzymatic hydrolysis (step 6 above), thereby recycling enzymes. In another the bottoms are returned to washing (step 5 above).
9) Stripping cooking alcohol and volatile byproducts from the washing step filtrate termed “spent liquor”.
10) Removing resinous wood components from the stripped spent liquor by skimming.
11) Autohydrolyzing spent liquor hemicelluloses by heating the stripped and skimmed spent liquor to form hemicellulosic hydrolyzate.
12) Optionally, filtering insoluble lignin from the hemicellulosic hydrolyzate.
13) Optionally, reacting filtered insoluble lignin with a sulfite-base chemical to convert it to lignosulfonate with counter ion.
14) Neutralizing the filtered hemicellulosic hydrolyzate with an alkaline chemical.
15) Fermenting hemicellulosic hydrolyzate to fermented beer using pentose utilizing micro-organism.
16) Distilling hemicellulosic alcohol from the fermented beer.
17) Concentrating distillation bottoms to recover soluble lignosulfonate.
18) Combusting excess lignosulfonate to produce process energy.

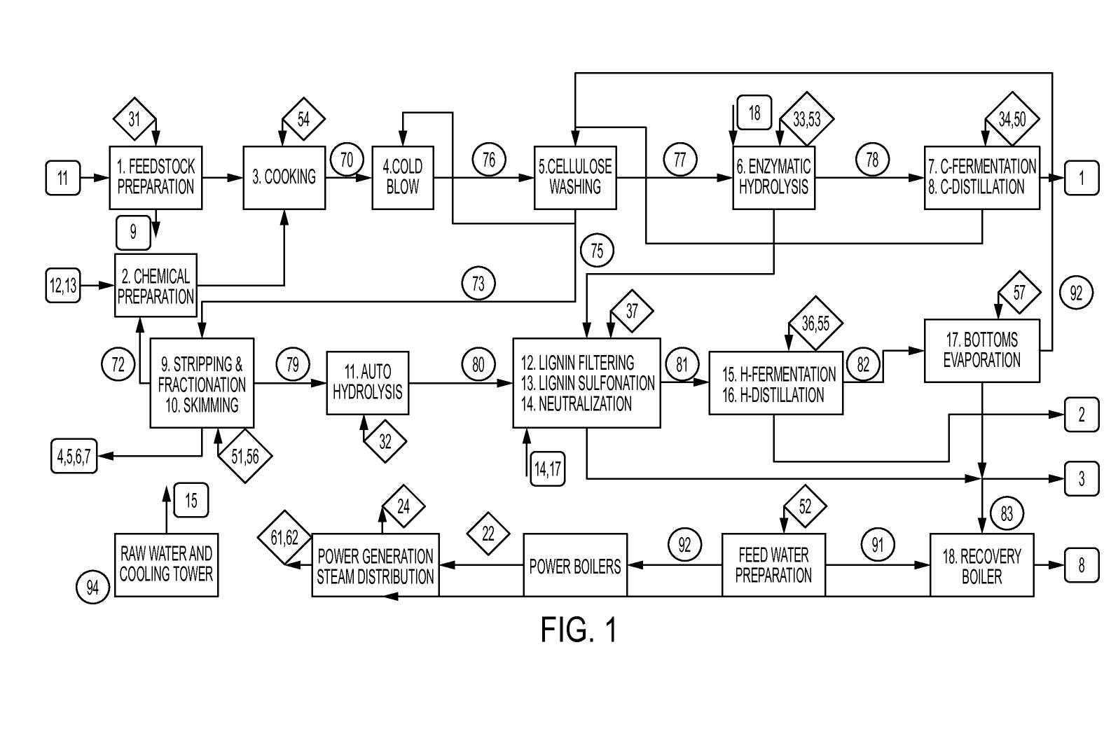

The first process step is “feedstock preparation”, element 1 in FIG. 1, in which the lignocellulosic material feedstock (stream 1) is comminuted in small pieces. The feedstock may be debarked, if appropriate, and washed from dirt (9). The feedstock may be preheated using hot water or steam (31) in a preheater vessel prior to the cooking vessel. The transfer from the preheater vessel to the cooking vessel is performed using a compaction screw or high pressure lock feeder or alternate device to produce a high pressure plug.

The second process step is “chemical preparation”. The alcohol from recovery stripper (72) is condensed at high concentration. Recovered SO₂ (74) is stripped to high strength and compressed to liquid form. Reacted and lost sulfur dioxide is replaced from liquid storage (12) or sulfur burner via a scrubber. The mixture is adjusted to cooking strength with makeup alcohol and/or water (13). These cooking chemicals are metered and mixed to predetermined ratio (71). Typical alcohol, water, and sulfur dioxide ratios by weight are 25-75% of both alcohol and water, and 9-50% of sulfur dioxide, and preferably 40% alcohol, 40% water and 20% sulfur dioxide; this solution is termed cooking liquor. The alcohol is from a group of aliphatic alcohols; methanol, ethanol, propanol and butanol. The cooking liquor is added to the lignocellulosic material in the cooking vessel. The lignocellulosic material to cooking liquor ratio is varied between 1:1 to 1:4, for example, 1:1, 1:2, 1:3, or 1:4, and preferably 1:2. In an alternative method, the cooking may be performed in less than 9% SO₂ in vapor phase reactor or in liquid phase reactor as described in United States Patent Application 20070254348 (Retsina; et al., Nov. 1, 2007) and United States Patent Application 20090236060 (Retsina; et al., Sep. 24, 2009).

The third process step is “cooking”. Steam (54) is used in the cooking vessel to heat the lignocellulosic material for a predetermined time of 10 minutes or more. Most of the lignin and hemicelluloses are dissolved. Cellulose is separated, but remains resistant to hydrolysis. Lignin is partially sulfonated, rendering it to a soluble form. Depending on the lignocellulosic material to be processed, the cooking conditions are varied, with temperatures from 65° C. to 160° C. or more, preferably 140° C., and corresponding pressures from 1 atmosphere to 20 atmospheres.

The fourth process step is “Cold Blow”, where the cooked lignocellulosic material (70) is cooled with countercurrent wash filtrate (75). The liquor pressure is reduced in an external flash tank to release SO₂. The cellulose (76) is then sent to washing in the fifth process step.

The fifth process step is “cellulose washing”, where filtrate termed “spent liquor” is removed (75) from the cellulose. The washing proceeds counter currently so that the highest solids and alcohol concentration contacts the cellulose from the cooking vessel first. The washing sequence may consist of pressure diffusers, screw presses, wash presses, drum washers, centrifuges and distillation columns. The distillation column may be used to recover alcohol from wash filtrate. The strongest filtrate is sent for cold blow dilution and for stripping (73). Washed cellulose goes to enzymatic hydrolysis step (77).

The sixth process step is “enzymatic hydrolysis”, where washed cellulose is mixed with enzymes (18). The enzymes may be dewatered to reduce the volume of the enzymatic hydrolysis holding tank size. An existing pulp decker or paper machine fourdrinier section may be used for dewatering. The enzyme mixing and holding may be repeated one or more times. Finally, the solid lignin (84) may be filtered out from the resulting cellulosic hydrolyzate and be sent to the autohydrolysis step.

The seventh process step is “cellulosic fermentation”, where micro-organism are added to the cellulosic hydrolyzate to convert it to cellulosic alcohol.

The eight process step is “cellulosic distillation”, where cellulosic alcohol is concentrated and purified (1). The distillation is performed with steam (34,50). The alcohol purification step may be combined with hemicellulosic fermentation. The bottoms of the distillation are sent to cellulose washing to recover unfermented pentoses. This step may also be practiced separately from the hemicellulosic sugar distillation. In that case distillation is practiced at low temperature and part of the distillation bottoms, containing yeast and enzymes, is recycled back to the enzymatic hydrolysis step.

The ninth process step is “stripping and fractionation”, where the cooking alcohol is removed from the spent liquor. The stripping column system may also remove other volatile byproducts from cooking step, including methanol, furfural, and acetic acid (4,5,6). This step may also include concentration of the cooking liquor. The concentrated alcohol (72) is sent to chemical preparation.

The tenth process step is “resin skimming”, where resinous water insoluble material (7) is skimmed from top of the stripped spent liquor. This step is necessary especially for pine, which contains pinosylvin and other resins.

The eleventh process step is “autohydrolysis”, where stripped and skimmed spent liquor is heated with steam (32) in a reactor to hydrolyze its hemicellulosic sugars (79) to hemicellulosic hydrolyzate. Reaction temperature is between 100° C. and 200° C. and the reaction time is between 2 minutes and 4 hours. Lignin from enzymatic hydrolysis (84) may be added to the reactor.

The twelfth process step is “lignin filtering”, where insoluble lignin is removed from the hemicellulosic hydrolyzate (80). This step may be combined with removal and washing of insoluble lime, if it is used for neutralization. The lignin is washed and dewatered to a high concentration to avoid sugar losses. This step is optional.

The thirteenth process step is “lignin sulfonation”, where lignin is rendered to soluble form by heating it with steam (37) in the presence of bisulfate ion. In the preferred embodiment of the invention, this is calcium sulfite (14) based lignosulfonate (3). Use of magnesium, sodium, and ammonium bases are also possible. This step is optional.

The fourteenth process step is “neutralization”. Insoluble base, for example lime (17), may be used for neutralizing the soluble lignosulfonic acids. The resulting insoluble calcium sulfite may be recycled to lignin sulfonation step. Other soluble bases, for example ammonium hydroxide and magnesium oxide, may be carried through fermentation, distillation and concentration. Optionally any precipitate may be removed at this step.

The fifteenth process step is “hemicellulosic fermentation”, where hemicellulosic hydrolyzate (81) is converted to hemicellulosic alcohol using an organism that can convert pentose and hexose sugars.

The sixteenth process step is “hemicellulosic distillation”, where hemicellulosic alcohol is concentrated and purified (2). The alcohol purification step may be combined with hemicellulosic fermentation. The bottoms of the fermentation (82) are sent to bottoms concentration step.

The seventeenth process step is “bottoms concentration”, where lignosulfonates and unfermented hemicelluloses are concentrated. The concentration step may be performed by steam (57) evaporation or by membrane separation. This lignosulfonate product (83) may be burned or sold to market as slurry or dried product.

The eighteenth process step is “Recovery Boiler”, where concentrated organics (83) are combusted to create process energy. The combustion step may be eliminated, if alternate use for lignosulfonates is available. The combustion is preferably performed in fluidized bed reactor, and, optionally, SO₂ is recovered from flue gases by scrubbing.

In addition the process includes process steam plant to provide process steam (61,62) and steam (22) to produce electricity (24). The water plant to provides process water (15) and boiler water (91,92) as well as water cooling and wastewater (94) treatment plant. Process integration is practiced to minimize process energy requirement.

[Para 31] Although other modifications and changes may be suggested by those skilled in the art, it is the intention of the inventors to embody within the patent warranted hereon that all changes and modifications as reasonably and properly come within the scope of their contribution to the art.

1-31. (canceled)

32. A biorefining process for producing sugars from lignocellulosic biomass, said process comprising dissolving hemicelluloses and lignin from said lignocellulosic biomass to generate cellulose solids, in a cooking vessel, with a liquor comprising heated aqueous alcohol and sulfur dioxide, wherein lignosulfonic acids are produced during said dissolving; in a separate reactor, hydrolyzing said hemicelluloses with said lignosulfonic acids, to generate hemicellulose monomer sugars; and recovering or further processing said hemicellulose monomer sugars.

33. The process of claim 32, wherein said lignocellulosic biomass is contacted with steam or hot water prior to introduction of said lignocellulosic biomass into said cooking vessel.

34. The process of claim 32, wherein said liquor is present in a liquid phase.

35. The process of claim 32, wherein said liquor is present in a vapor phase.

36. The process of claim 32, wherein said alcohol is present in said liquor at a concentration of 25% or more by weight.

37. The process of claim 32, wherein said sulfur dioxide is present in said liquor at a concentration of 9% or more by weight.

38. The process of claim 37, wherein said sulfur dioxide is present in said liquor at a concentration of 50% or less by weight.

39. The process of claim 32, wherein said dissolving in said cooking vessel is conducted for at least 10 minutes and at a temperature from 65° C. to 160° C. or more.

40. The process of claim 32, said process further comprising recovering said sulfur dioxide and said alcohol from said liquor, and then reusing recovered sulfur dioxide and recovered alcohol in said cooking vessel.

41. The process of claim 40, wherein said recovered sulfur dioxide is compressed to liquid form.

42. The process of claim 32, said process further comprising countercurrently washing said cellulose solids.

43. The process of claim 42, wherein said process utilizes one or more pressure diffusers, screw presses, wash presses, drum washers, centrifuges, distillation columns, or combinations thereof.

44. The process of claim 32, wherein an alcohol stripper column is configured to treat one or more wash filtrates to remove said alcohol, and wherein alcohol stripper column bottoms are reused for washing of said cellulose solids.

45. The process of claim 32, wherein said hydrolyzing said hemicelluloses is conducted for at least 2 minutes at a reaction temperature from 100° C. to 200° C.

46. The process of claim 32, said process further comprising removing insoluble lignin after said hydrolyzing said hemicelluloses.

47. The process of claim 46, said process further comprising reacting said insoluble lignin with a sulfur-containing chemical to convert said lignin to a lignosulfonate.

48. The process of claim 32, said process further comprising enzymatically hydrolyzing said cellulose solids to produce glucose.

49. The process of claim 48, said process comprising recycling distillation bottoms containing enzymes to a unit for enzymatically hydrolyzing said cellulose solids, thereby recycling enzymes.

50. The process of claim 32, said process further comprising combusting organic residues to produce energy for said process.

51. The process of claim 32, said process further comprising combusting inorganic residues in a fluidized bed boiler, and recovering inorganic sulfur dioxide from a flue gas of said fluidized bed boiler.
